(19) United States
(12) Patent Application Publication
(10) Pub. No.: US 2011/0202024 A1
(43) Pub. Date: Aug. 18, 2011

(54) Catheter Leg Bag Support Device

(76) Inventor: Deirdre Lisa Cozzens, Ralston, WY (US)

(21) Appl. No.: 12/847,504

(22) Filed: Jul. 30, 2010

Related U.S. Application Data
(60) Provisional application No. 61/305,750, filed on Feb. 18, 2010.

(51) Int. Cl. A61F 5/44

(52) U.S. Cl. 604/327

(57) ABSTRACT

A catheter leg bag support system is provided for supporting and holding in place a catheter bag. An adjustable belt is secured around a user's waist, with a drop down strap having an adjustable length attached to the belt. A hook and loop fastener wrap is provided on the drop down strap to attach to excessive catheter tubing in order to keep the tubing in a desired position. A reinforced stabilizer member is attached to the drop down strap to support a catheter bag, and can keep the catheter bag flat. Button posts attached to the reinforced member can quickly and easily connect to a conventional catheter leg bag. The catheter bag is secured in place along a user's leg using a soft and stretchable band that can be adjusted as loose or tight as desired.

### CROSS REFERENCE TO RELATED APPLICATION

Priority of U.S. Provisional Patent Application Ser. No. 61/305,750 filed Feb. 18, 2010, incorporated herein by reference, is hereby claimed.

### STATEMENTS AS TO THE RIGHTS TO THE INVENTION MADE UNDER FEDERALLY SPONSORED RESEARCH AND DEVELOPMENT

None

### BACKGROUND OF THE INVENTION

1. Field of the Invention

This application relates to a catheter leg bag support apparatus, specifically an apparatus for holding and securing a catheter leg bag in place.

2. Brief Description of the Prior Art

Physically active individuals that have to wear a catheter leg bag are confronted with the problem of how to hold the bag up. The bags are typically supplied with two straps, one for the top of the bag and one for the bottom of the bag. These conventional straps are typically stretchable to fit around a user's leg. However, due to legs being tapered there is no way that such a bag will stay in place using such conventional straps. Therefore as the urine fills in the leg bag the straps cannot hold up the bag they twist and pinch as the bag slips down the leg, and as a result of the bag falling it pulls on the catheter tube which causes a great deal of pain to the user.

Several types of supports have been designed to hold up the bags worn on the leg. However, such supports are heavy, intrusive, clumsy, and complicated. Many prior art supports require great dexterity from a user, which is something many individuals wearing catheters do not have.

### SUMMARY OF THE PRESENT INVENTION

The present invention provides a catheter leg bag support system that will solve the issues a catheter bag wearer faces. In the preferred embodiment, a fully adjustable waist belt is provided having an easy release buckle. A flat drop down strap can be raised to the upper thigh or lowered to the ankle as needed for the particular individual. The drop down strap has a hook and loop fastener tab (for example, a Velcro® brand fastener) tab so the excess tubing can be kept out of the way so it won't get caught on clothing or otherwise be pulled painfully. A stabilizer member keeps the bag flat and straight, so bunching won't occur which causes the inflow of urine to be slowed or stopped. Button posts that are attached to the stabilizer member make it easy to attach any leg bag and hold the bag securely. Other objects, features, and advantages of the invention will be clear from the included drawings.

### BRIEF DESCRIPTION OF THE DRAWINGS

The foregoing summary, as well as the following detailed description of the preferred embodiments, is better understood when read in conjunction with the appended drawings. For the purpose of illustrating the invention, the drawings show certain preferred embodiments. It is understood, however, that the invention is not limited to the specific methods and devices disclosed.

FIG. 1 depicts a perspective view of the catheter leg bag support apparatus of the present invention.

FIG. 2 depicts a front view of a belt with easy release buckle and ladder configuration of the drop down strap of the present invention.

FIG. 3 depicts a view of drop down strap and hook and loop fastener tab of the present invention.

FIG. 4 depicts a front view of reinforced stabilizer member and button posts of the present invention.

FIG. 5 depicts a detailed view of button posts of the present invention.

FIG. 6 depicts a detailed view of a stretchable leg strap with band loop holder of the present invention.

FIG. 7 depicts a view of the drop down strap of the present invention with ladder for adjustment of said strap.

### DETAILED DESCRIPTION OF A PREFERRED EMBODIMENT OF THE INVENTION

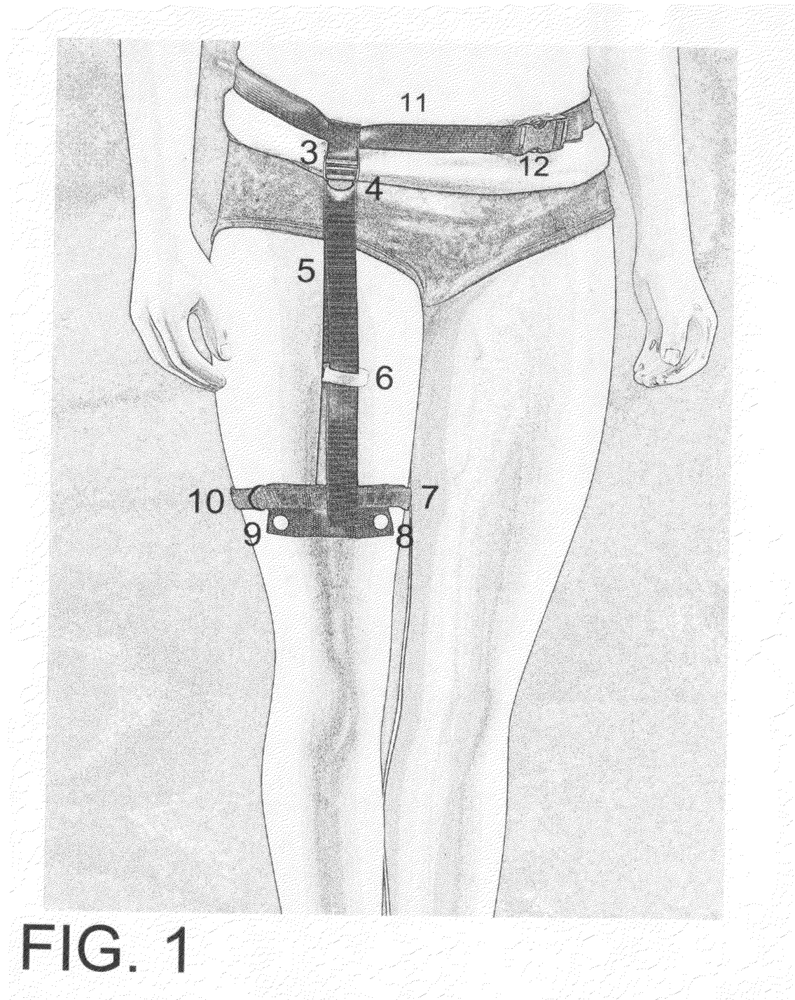

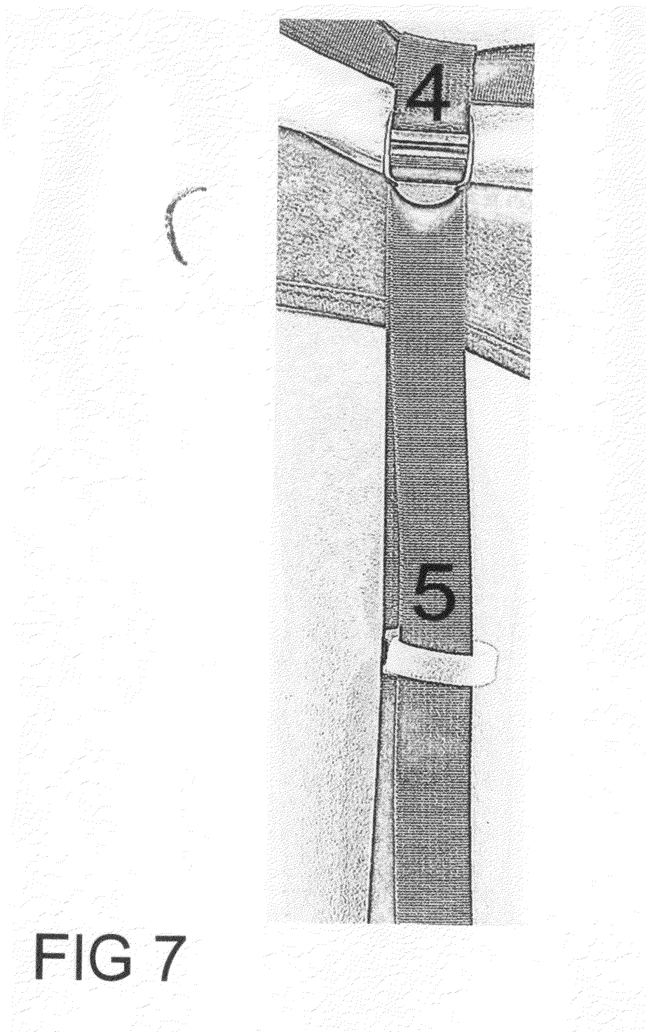

FIG. 1 depicts a perspective view of the catheter leg bag apparatus of the present invention. Referring to FIG. 1, waist belt 11 is worn around a user's waist and secured in place using snap-able strap bracket 12 having an adjustable strap buckle for adjusting the length of waist belt 11. Adjustable drop down strap 5 is attached to said waist belt 11 via snap-able strap bracket 3 having an adjustable strap buckle and connection loop 4 (depicted in FIG. 7). Although drop down strap 5 can be positioned in many different locations according to a user's comfort and individual preference, as depicted in FIG. 1, drop down strap 5 can be beneficially oriented so that it extends down the front portion of a user's thigh.

Still referring to FIG. 1, in the preferred embodiment, drop down strap 5 is folded to provide a “ladder” configuration with the adjustable buckle of snap-able strap bracket 3. Such ladder configuration and adjustable buckle for connection to strap bracket 3 allows drop down strap 5 of the present invention to be lowered or raised in order to position a catheter bag at a desired location on a user's leg. Although such catheter bag can be positioned in many different locations according to a user's comfort and individual preference, typically such bag is positioned at the upper portion of a user's thigh or the lower portion of a user's calf. In this configuration, drop down strap 5 and snap-able strap bracket 3, as well as other components attached thereto, can remain secure in virtually any location.

Still referring to FIG. 1, hook and loop fastener tab 6 permits a user to secure excessive catheter tubing in a desired location such as, for example, away from clothes. Said fastener tab 6 also helps to reduce and/or eliminate pulling forces on said catheter tubing which, in turn, can result in painful pulling on a user's internal catheter components.

Leg band 7 can be disposed around a user's leg at a desired location. In the preferred embodiment, leg band 7 is beneficially constructed of soft, stretchable material. If desired, a strap or tube can be wrapped around the back of a leg and through stretchable loop band holder 10 so that such strap or tube does not get twisted or climb up a user's leg. In the preferred embodiment, reinforced stabilizer member 9 provides a substantially stiff platform or surface for supporting a catheter bag, while remaining generally flat and smooth so that said bag will not pucker and restrict the flow of urine into the bag. Reinforced stabilizer member 9 is also strong enough to support the weight of catheter bags.

Still referring to FIG. 1, at least one button post 8 is provided to connect to a catheter leg bag during use, allowing such leg bag to rest securely in a desired position along a user's leg. Each of said at least one button post 8 has head dimensions (including, without limitation, diameter) such that a catheter bag can be quickly and easily attached to said button post(s) 8, but will not slip off during use. In the preferred embodiment, said at least one button post 8 is long enough to accommodate the thickness of virtually any catheter bag.

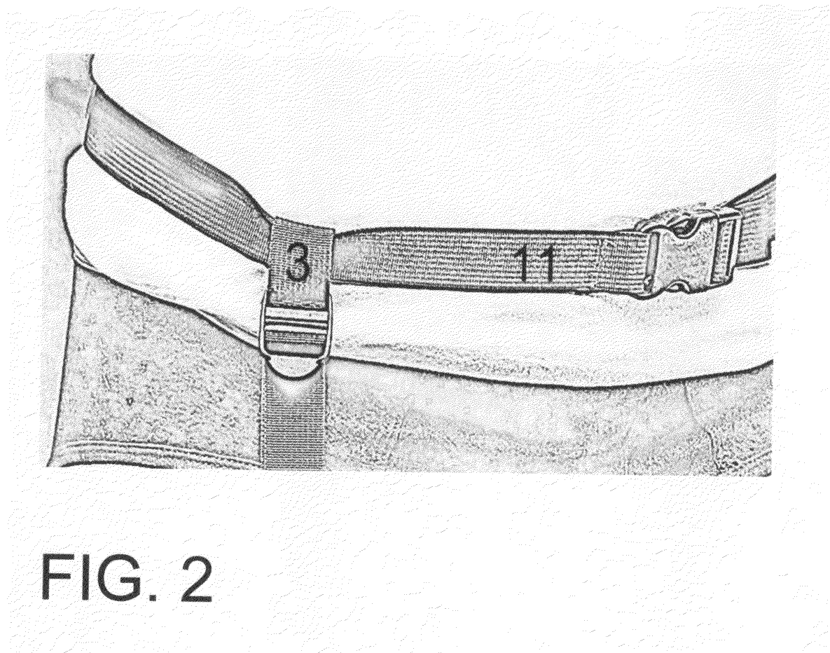

FIG. 2 depicts waist belt 11 and easy release snap-able bracket 3 having an adjustable strap buckle for adjustably securing drop down strap 5 in a desired position.

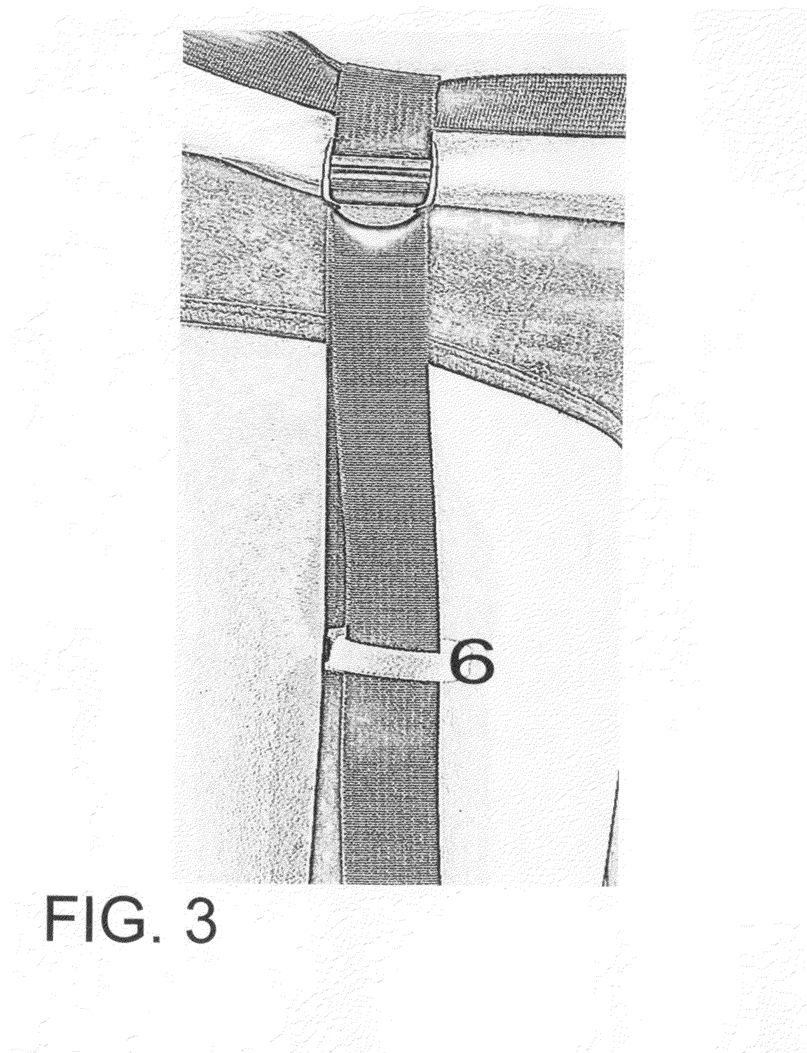

FIG. 3 depicts drop down strap 5 with hook and loop fastener tab 6 for the attachment to excess catheter tubing. Hook and loop fastener tab 6 can be opened and then attached to itself, and is easy to open, close and otherwise manipulate for users having dexterity issues.

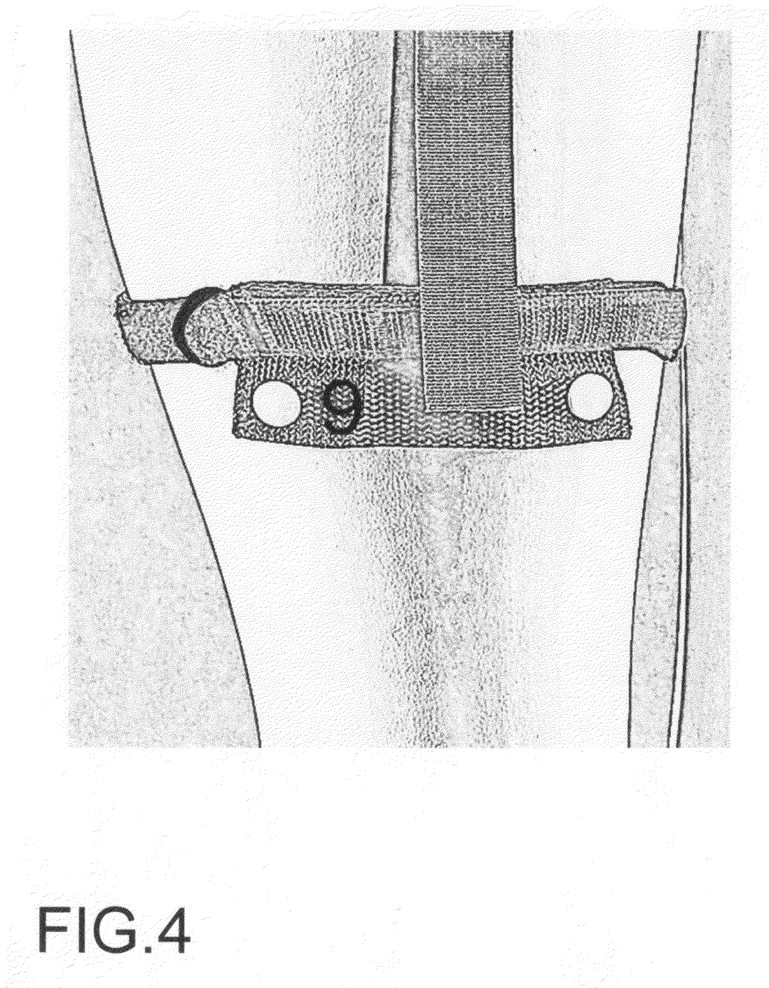

FIG. 4 depicts reinforced stabilizer member 9 having button posts 8. Reinforced stabilizer member 9 can beneficially hold a catheter leg bag substantially flat across the top of said bag to permit urine to flow unrestricted from the catheter tube into said bag. Said reinforced stabilizer member 9 prevents the bag from bunching or puckering, in order to permit free flow into a catheter bag.

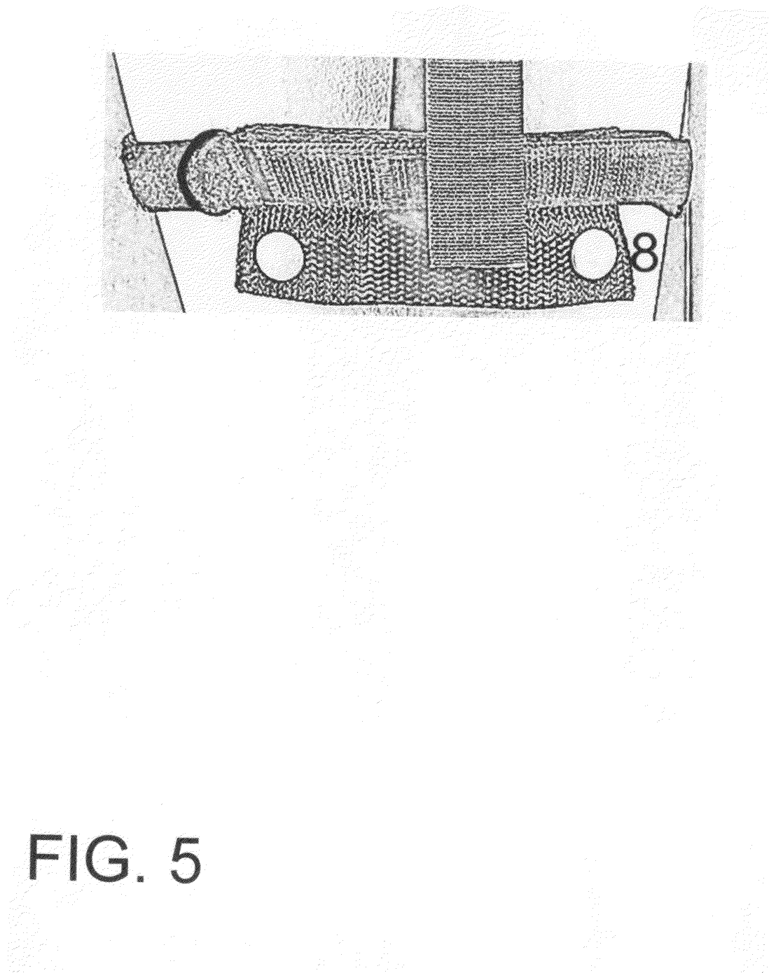

FIG. 5 depicts a detailed view of button posts 8. In the preferred embodiment, said button posts 8 are beneficially positioned at a desired location to support catheter bag, while positioning such bag firmly across a leg. Button posts 8 permit fast and easy connection and disconnection of most conventional catheter leg bags via holes or slots built into said catheter leg bags.

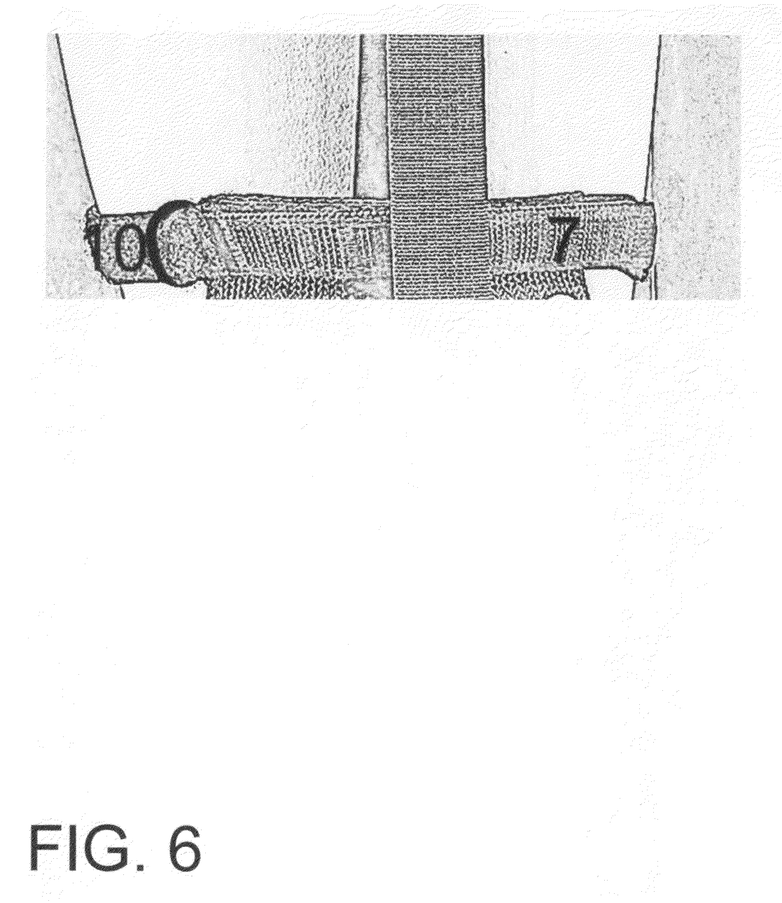

FIG. 6 depicts a detailed view of leg band 7 and stretchable band loop holder 10 of the present invention. Such components are ideally constructed of soft and stretchable fabric so as not to irritate a user's skin. The length of leg band 7 is adjustable, so that said leg band 7 can be secured as loosely or tightly as a user desires, without irritating delicate skin.

FIG. 7 depicts a side view of adjustable drop down strap 5 of the present invention. Adjustable drop down strap 5 is attached to said waist belt 11 via snap-able strap bracket 3 having an adjustable strap buckle, and connection loop 4. In the preferred embodiment, drop down strap 5 is folded and has enough strap length to adjust to any length that a user might need in different situations. Drop down strap 5 can be quickly adjusted up or down, as needed, and can (along with the other components of the present invention) support the weight of virtually any catheter leg bag.

From the description above, a number of advantages of some embodiments of the catheter leg bag support apparatus of the present invention are evident including, without limitation, the following:

a) The belt of the present invention is lightweight so that a wearer is not burdened with a heavy, bulky or complicated apparatus;

b) The attachment means and other components of the catheter bag support apparatus of the present invention are easy to operate for any wearer;

c) The catheter bag support apparatus of the present invention can hold a catheter leg bag securely in a desired position, and such bag can stay in place for extended periods of time;

d) The catheter bag support apparatus of the present invention can be easily used by men, women and children;

e) The catheter bag support apparatus of the present invention has no pinching, twisting or falling leg straps;

f) The catheter bag support apparatus of the present invention does not require a specialty catheter bag;

g) The catheter bag support apparatus of the present invention is durable; and

h) A hook and loop fastener tab keeps tubing from bunching up under clothes and getting kinked during use of the catheter bag support apparatus of the present invention.

The above-described invention has a number of particular features that should preferably be employed in combination, although each is useful separately without departure from the scope of the invention. While the preferred embodiment of the present invention is shown and described herein, it will be understood that the invention may be embodied otherwise than herein specifically illustrated or described, and that certain changes in form and arrangement of parts and the specific manner of practicing the invention may be made within the underlying idea or principles of the invention.

1. A catheter leg bag support apparatus comprising:
a. a waist belt having a side release buckle;
b. a loop slidably disposed on said waist belt;
c. a strap having a first end, a second end and a length, wherein said first end is attached to said loop;
d. a stretchable band connected to the second end of said strap;
e. a substantially stiff member connected to said stretchable band; and
f. at least one button post attached to said substantially stiff member.

2. The support device of claim 1 wherein wearers bag is supported by a waist belt

3. The support device of claim 1 wherein wearer of bag has a drop down strap with a one wrap velcro strap sewn on strap for controlling excess tubing

4. The support device of claim 1 wherein the wearer has a patch sewn to the drop down strap FIG. 1 #9 with button posts to hang any catheter bag to

5. The catheter leg bag support apparatus of claim 1, wherein said stretchable band is oriented substantially perpendicular to said strap.

6. The catheter leg bag support apparatus of claim 1, further comprising a wrap band disposed around said strap.

7. The catheter leg bag support apparatus of claim 6, wherein said wrap band further comprises a hook and loop fastener.

8. A catheter leg bag support comprising:
a. a waist belt having a side release buckle;
b. a loop slidably disposed on said waist belt;
c. a ladderlock bracket connected to said loop;
d. a strap having a first end, a second end and a length, wherein said first end is attached to said ladderlock bracket;
e. a stretchable band connected to the second end of said strap;
f. a substantially stiff member connected to said stretchable band; and
g. at least one button post attached to said substantially stiff member.

9. The catheter leg bag support apparatus of claim 1, wherein said stretchable band is oriented substantially perpendicular to said strap.

10. The catheter leg bag support apparatus of claim 8, further comprising a wrap band disposed around said strap.

11. The catheter leg bag support apparatus of claim 10, wherein said wrap band further comprises a hook and loop fastener.
